(19) United States
(12) Patent Application Publication
(10) Pub. No.: US 2023/0139627 A1
(43) Pub. Date: May 4, 2023

(54) METHOD FOR DETERMINING PSYCHOLOGICAL STRESS

(71) Applicant: bOMDIC, Inc., New Taipei (TW)

(72) Inventors: WEI-CHIAO CHANG, New Taipei (TW); YU-TING LIU, New Taipei (TW); AMY PEI-LING CHIU, New Taipei (TW)

(21) Appl. No.: 17/518,550

(22) Filed: Nov. 3, 2021

(51) Int. Cl. A61B 5/16; A61B 5/00; A61B 5/024; A61B 5/0245; G16H 10/60; G16H 40/67; G16H 50/30

(52) U.S. Cl. CPC A61B 5/165; A61B 5/7221; A61B 5/742; A61B 5/02405; A61B 5/0245; G16H 10/60; G16H 40/67; G16H 50/30

(57) ABSTRACT

The present invention discloses a method for determining psychological stress. The method includes the steps of: acquiring physiological parameter data comprising a first physiological parameter and a second physiological parameter temporally corresponding to the first physiological parameter; determining a reliability in an estimation of the psychological stress of the physiological parameter data based on a degree of match between the physiological parameter data and a plurality of criteria, wherein the plurality of criteria are determined based on a first reference value of the first physiological parameter and a second reference value of the second physiological parameter; and determining an indicator of the psychological stress based on the physiological parameter data and the reliability in the estimation of the psychological stress of the physiological parameter data.

### BACKGROUND OF THE INVENTION

### 1. Field of the Invention

The present invention relates to a method for monitoring the physiological state of the user, and more particularly to a method for monitoring the physiological state by determining a psychological stress.

### 2. Description of Related Art

The psychological stress can be defined as any type of change that causes physical, emotional, or psychological strain. Stress is your body's response to anything that requires attention or action. The psychological stress may be referred to as the mental stress. Lower psychological stress may be desired, beneficial, and even healthy. Positive psychological stress helps improve athletic performance. It also plays an important role in motivation, adaptation, and reaction to the environment. However, higher stress may lead to bodily harm. Stress can increase the risk of strokes, heart attacks and mental illnesses (e.g., depression or anxiety). When the psychological stress exceeds psychological endurance of the human body, psychological imbalance occurs, and the psychological disease is caused. Therefore, a measurement of the psychological stress is always an important topic.

For evaluating the psychological stress, a subjective self-evaluation method (e.g., questionnaire), a face-to-face conversation and observation of professional psychologist need to be combined. However, it is not suitable to continuously evaluate psychological stress in real time.

Conventionally, the heart rate variation (e.g., the standard deviation of the beat-to-beat interval extracted from the electrocardiogram (ECG) signal)) or the relative heart rate variation determined based on a reference value of the heart rate variation is used to estimate the psychological stress. The psychological stress increases as each the heart rate variation and the relative heart rate variation decreases. However, even if there is the same heart rate variation or the same relative heart rate variation in two psychological states of the user, there isn't necessarily the same psychological stress sensed by the user in two psychological states.

Accordingly, the present invention proposes a method for determining a psychological stress to overcome the above-mentioned disadvantages.

### SUMMARY OF THE INVENTION

In the present invention, at least two physiological parameters (e.g., a first physiological parameter and a second physiological parameter) of the physiological parameter data are used together to determine a psychological stress. Preferably, the first physiological parameter is a heart rate variation. Another physiological parameter (e.g., second physiological parameter) is taken into account to reduce imprecision of an estimation of the psychological stress determined by one physiological parameter (e.g., heart rate variation).

When at least two physiological parameters (e.g., a first physiological parameter and a second physiological parameter) of the physiological parameter data are used together to determine a psychological stress, there is a different reliability in an estimation of the psychological stress for the physiological parameter data.

In the present invention, an algorithm is performed to determine the psychological stress based on the reliability in an estimation of the psychological stress of the physiological parameter data such that the difference between the psychological stress determined in the higher reliability in an estimation of the psychological stress and the psychological stress determined in the lower reliability in an estimation of the psychological stress can meet the actual difference of the psychological stress more. Therefore, a modified algorithm determined based on the reliability in an estimation of the psychological stress can increase a precision of the estimation of the psychological stress.

Besides, even if the psychological stress is estimated to be the same in an early time and in a later time for a person, the person doesn't necessarily sense the same psychological stress. The person may actually sense the different psychological stress because the reference value of each of at least two physiological parameters used in an estimation of the psychological stress may vary with time. In the present invention, the reference value of each of at least two physiological parameters is determined based on the corresponding history data so as to increase a precision of the estimation of the psychological stress.

By the algorithm implemented in the computer of the present invention, the computer of the present invention performs operations described in claims or the following descriptions to determine the psychological stress.

In one embodiment, the present invention discloses a method for determining a psychological stress. The method comprises: acquiring physiological parameter data measured by at least one sensor, wherein the physiological parameter data comprises a first physiological parameter and a second physiological parameter temporally corresponding to the first physiological parameter; determining, by a processing unit, a reliability in an estimation of the psychological stress of the physiological parameter data based on a degree of match between the physiological parameter data and a plurality of criteria, wherein the plurality of criteria are determined based on a first reference value of the first physiological parameter and a second reference value of the second physiological parameter; and determining, by the processing unit, an indicator of the psychological stress based on the physiological parameter data and the reliability in the estimation of the psychological stress of the physiological parameter data.

In one embodiment, the present invention discloses a method for determining a psychological stress. The method comprises: acquiring physiological parameter data measured by at least one sensor, wherein the physiological parameter data comprises a first physiological parameter and a second physiological parameter temporally corresponding to the first physiological parameter, wherein the first physiological parameter is a heart rate variation and the second physiological parameter is a heart rate; determining, by a processing unit, a reliability in an estimation of the psychological stress of the physiological parameter data based on a degree of match between the physiological parameter data and a plurality of criteria, wherein the plurality of criteria are determined based on a first reference value of the first physiological parameter and a second reference value of the second physiological parameter; and determining, by the processing unit, an indicator of the psychological stress based on the physiological parameter data and the reliability in the estimation of the psychological stress of the physiological parameter data.

In one embodiment, the present invention discloses a method for determining a psychological stress. The method comprises: acquiring physiological parameter data measured by at least one sensor, wherein the physiological parameter data comprises a first physiological parameter and a second physiological parameter temporally corresponding to the first physiological parameter, wherein the first physiological parameter is a heart rate variation and the second physiological parameter is a heart rate; determining, by a processing unit, a reliability in an estimation of the psychological stress of the physiological parameter data based on a degree of match between the physiological parameter data and a plurality of criteria, wherein the plurality of criteria are determined based on a first reference value of the first physiological parameter and a second reference value of the second physiological parameter; modifying at least one of a first relative physiological parameter and a second relative physiological parameter based on the reliability in the estimation of the psychological stress of the physiological parameter data to determine at least one modified physiological parameter, wherein the first relative physiological parameter is a first difference between the first physiological parameter and the first reference value of the first physiological parameter, and the second relative physiological parameter is a second difference between the second physiological parameter and the second reference value of the second physiological parameter, wherein one of the at least one modified physiological parameter is determined based on a combination of a weighting factor and corresponding one of the first relative physiological parameter and the second relative physiological parameter, wherein the weighting factor is adjusted based on the reliability in the estimation of the psychological stress of the physiological parameter data; and determining the indicator of the psychological stress based on the at least one modified physiological parameter.

The detailed technology and above preferred embodiments implemented for the present invention are described in the following paragraphs accompanying the appended drawings for people skilled in the art to well appreciate the features of the claimed invention.

### BRIEF DESCRIPTION OF THE DRAWINGS

The foregoing aspects and many of the accompanying advantages of this invention will become more readily appreciated as the same becomes better understood by reference to the following detailed description when taken in conjunction with the accompanying drawings, wherein:

FIG. 1 illustrates a schematic block diagram of an exemplary apparatus in the present invention; and

FIG. 2 illustrates a method for determining a psychological stress.

### DETAILED DESCRIPTION OF THE ILLUSTRATED EMBODIMENTS

The detailed explanation of the present invention is described as following. The described preferred embodiments are presented for purposes of illustrations and description and they are not intended to limit the scope of the present invention.

Activity Intensity

The activity intensity may refer to how much energy is expended when taking activity. The activity intensity may define how hard the body has to work to overcome an activity/exercise. The activity intensity may be measured in the form of the internal workload. The parameter of the activity intensity associated with the internal workload may be associated with a heart rate, an oxygen consumption, a pulse, a respiration rate and RPE (rating perceived exertion). The activity intensity may be measured in the form of the external workload. The parameter of the activity intensity associated with the external workload may be associated with a speed, an acceleration, a power, a force, an energy expenditure rate, a motion intensity, a motion cadence or other kinetic data created by the external workload resulting in energy expenditure. The heart rate may be often used as a parameter of the activity intensity.

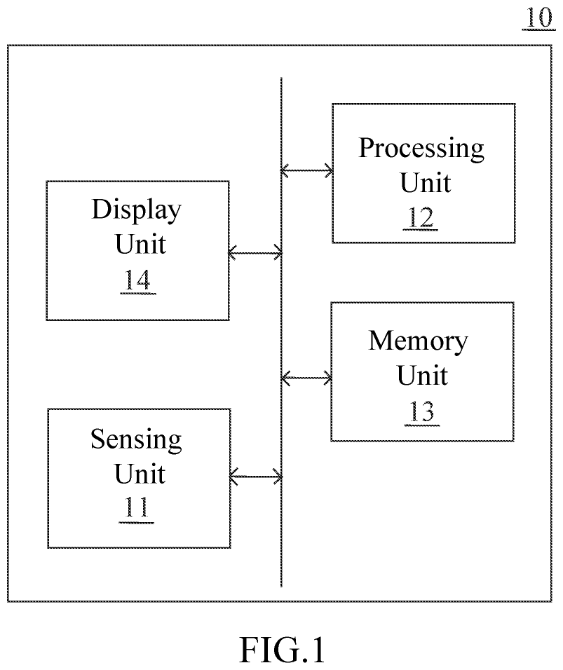

The method in the present invention may be applied in all kinds of apparatuses, such as a measurement system, the device worn on the individual (e.g., the device attached to the wrist belt or chest belt), a wrist top device, a mobile device, a portable device, a personal computer, a server or a combination thereof. FIG. 1 illustrates a schematic block diagram of an exemplary apparatus 10 in the present invention. The apparatus 10 may comprise a sensing unit 11 (e.g., at least one sensor), a processing unit 12, a memory unit 13 and a displaying unit 14. One unit may communicate with another unit in a wired or wireless way. The apparatus 10 may comprise at least one device; the sensing unit 11 may be in one device (e.g., the device worn on the individual or watch) and the processing unit 12 may be in another device (e.g., mobile device or mobile phone); the sensing unit 11 and the processing unit 12 may be in a single device (e.g., the device worn on the individual or watch). The sensing unit 11 may be attached to/comprised in a belt worn on the individual. The sensing unit 11 may be a sensor (e.g., heart activity sensor) which may measure a signal associated with the physiological data, the cardiovascular data or the internal workload from the person's body. The signal may be measured by applying a skin contact from chest, wrist or any other human part. The processing unit 12 may be any suitable processing device for executing software instructions, such as a central processing unit (CPU). The processing unit 12 may be a computing unit. The apparatus 10 may comprise at least one device; a first portion of the computing unit may be in one device (e.g., the device worn on the individual or watch), a second portion of the computing unit may be in another device (e.g., mobile device or mobile phone) and a first portion of the computing unit may communicate with a second portion of the computing unit in a wired or wireless way; a first portion of the computing unit and a second portion of the computing unit may be in a single device (e.g., the device worn on the individual or watch). The memory unit 13 may include random access memory (RAM) and read only memory (ROM), but it is not limited to this case. The memory unit 13 may include any suitable non-transitory computer readable medium, such as ROM, CD-ROM, DVD-ROM and so on. Also, the non-transitory computer readable medium is a tangible medium. The non-transitory computer readable medium includes a computer program code which, when executed by the processing unit 12, causes the apparatus 10 to perform desired operations (e.g., operations listed in claims). The display unit 14 may be a display for displaying an estimation of (an indicator of) the psychological stress. Optionally, the first reference value of the first physiological parameter and the second reference value of the second physiological parameter are also displayed. The displaying mode may be in the form of words, a voice or an image. The sensing unit 11, the processing unit 12, the memory unit 13 and the displaying unit 14 in the apparatus 10 may have any suitable configuration and it doesn't be described in detail therein.

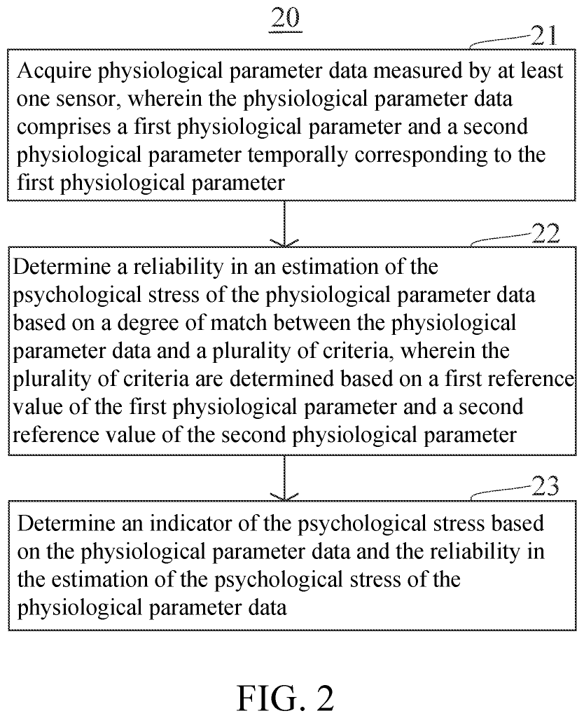

FIG. 2 illustrates a method 20 for determining a psychological stress. The method comprises:

Step 21: acquire physiological parameter data measured by at least one sensor 11, wherein the physiological parameter data comprises a first physiological parameter and a second physiological parameter temporally corresponding to the first physiological parameter;

Step 22: determine a reliability in an estimation of the psychological stress of the physiological parameter data based on a degree of match between the physiological parameter data and a plurality of criteria, wherein the plurality of criteria are determined based on a first reference value of the first physiological parameter and a second reference value of the second physiological parameter (by the processing unit 12);

Step 23: determine an indicator of the psychological stress based on the physiological parameter data and the reliability in the estimation of the psychological stress of the physiological parameter data (by the processing unit 12).

The first physiological parameter acquired in step 21 may be a heart activity parameter. The heart activity parameter may be associated with a plurality of beats and a plurality of beat intervals alternating with a plurality of beats; further, the heart activity parameter may be a time of the beat interval, such as RRI (RR interval: beat-to-beat interval extracted from the electrocardiogram (ECG) signal) or PPI (PP interval: beat-to-beat interval extracted from the photoplethysmography (PPG) signal). The heart activity parameter may be a parameter associated with a EDR (ECG-Derived Respiration) signal or a PDR (PPG-Derived Respiration) signal. The heart activity parameter may be a pulse rate. The heart activity parameter may be a heart rate. The heart rate may be in the form of a time of the beat interval (e.g., RRI or PPI). The heart rate may be in the form of BPM (beats per minute). The heart activity parameter may be a derivative of the previous heart activity parameter or the heart activity parameter may be determined based on another heart activity parameter. For example, the heart rate variability (HRV) information integrates sympathetic and parasympathetic activity of the autonomic nervous system that varies with the degree of response to the physical activity and can be an effective indicator of the degree of response to the physical activity; the heart rate variability (HRV) analysis may be performed to further analyze the beat-to-beat interval to obtain the heart activity parameter, such as a time-domain HRV parameter, a frequency-domain HRV parameter or a non-linear HRV parameter. The time-domain HRV parameter may be determined based on statistics computed over RR interval or PP interval, such as the number of the intervals per epoch, the standard deviation of the interval or the mean interval. The time-domain HRV parameter may be a mean of the beat-to-beat interval, a heart rate, a standard deviation of the beat-to-beat interval (e.g., SDNN: Standard Deviation of Normal to Normal) or a root mean square of the adjacent intervals differences (e.g., RMSSD: Root Mean Square of the Successive Differences). The frequency-domain HRV parameter may be determined based on the power spectral analysis of the heart activity. The frequency-domain HRV parameter may be a low frequency range power (LFP), a high frequency range power (HFP) or a ratio (LF/HF) between a high frequency (HF) and a low frequency (LF). The non-linear HRV parameter may be an entropy that measures complexity or regularity of the heart activity.

The first physiological parameter acquired in step 21 may be a respiratory parameter (e.g., respiration rate), a blood pressure signal or a blood oxygen signal.

Preferably, the first physiological parameter is associated with a variation of a heart activity parameter or is a variation of a heart activity parameter. The variation of the heart activity parameter may be associated with a difference between a first value of the heart activity parameter and a second value of the heart activity parameter. The first value of the heart activity parameter may be adjacent to the second value of the heart activity parameter. The variation of the heart activity parameter may be associated with an average of the difference between the first value of the heart activity parameter and the second value of the heart activity parameter, such as root mean square of the adjacent intervals differences (e.g., RMSSD: Root Mean Square of the Successive Differences). The variation of the heart activity parameter may be associated with a difference between a value of the heart activity parameter and a reference value (e.g., the average value) of the heart activity parameter, such as standard deviation of the beat-to-beat interval (e.g., SDNN: Standard Deviation of Normal to Normal). For convenience of description, the first physiological parameter is a variation of a heart rate abbreviated as a heart rate variation (e.g., a root mean square of the adjacent intervals differences (e.g., RMSSD: Root Mean Square of the Successive Differences)); however, the present invention is not limited to this case.

The second physiological parameter acquired in step 21 may be an intensity associated with the internal workload. The intensity associated with the internal workload may be a heart activity parameter. The heart activity parameter may be associated with a plurality of beats and a plurality of beat intervals alternating with a plurality of beats; further, the heart activity parameter may be a time of the beat interval, such as RRI (RR interval: beat-to-beat interval extracted from the electrocardiogram (ECG) signal) or PPI (PP interval: beat-to-beat interval extracted from the photoplethysmography (PPG) signal). The heart activity parameter may be a parameter associated with a EDR (ECG-Derived Respiration) signal or a PDR (PPG-Derived Respiration) signal. The heart activity parameter may be a pulse rate. The heart activity parameter may be a heart rate. The heart rate may be in the form of a time of the beat interval (e.g., RRI or PPI). The heart rate may be in the form of beats per minute. The heart activity parameter may be a derivative of the previous heart activity parameter or the heart activity parameter may be determined based on another heart activity parameter. For example, the heart rate variability (HRV) information integrates sympathetic and parasympathetic activity of the autonomic nervous system that varies with the degree of response to the physical activity and can be an effective indicator of the degree of response to the physical activity; the heart rate variability (HRV) analysis may be performed to further analyze the beat-to-beat interval to obtain the heart activity parameter, such as a time-domain HRV parameter, a frequency-domain HRV parameter or a non-linear HRV parameter. The time-domain HRV parameter may be determined based on statistics computed over RR interval or PP interval, such as the number of the intervals per epoch, the standard deviation of the interval or the mean interval. The time-domain HRV parameter may be a mean of the beat-to-beat interval, a heart rate, a standard deviation of the beat-to-beat interval (e.g., SDNN: Standard Deviation of Normal to Normal) or a root mean square of the adjacent intervals differences (e.g., RMSSD: Root Mean Square of the Successive Differences). The frequency-domain HRV parameter may be determined based on the power spectral analysis of the heart activity. The frequency-domain HRV parameter may be a low frequency range power (LFP), a high frequency range power (HFP) or a ratio (LF/HF) between a high frequency (HF) and a low frequency (LF). The non-linear HRV parameter may be an entropy that measures complexity or regularity of the heart activity.

The second physiological parameter acquired in step 21 may be a respiratory parameter (e.g., respiration rate), a blood pressure signal or a blood oxygen signal.

For convenience of description, the second physiological parameter is a heart rate in the form of beats per minute; however, the present invention is not limited to this case.

The first physiological parameter and the second physiological parameter are measured by at least one sensor (e.g., heart activity sensor, heart rate sensor or the corresponding sensor for the measured physiological parameter). In one embodiment, the first physiological parameter may be measured by a first sensor and the second physiological parameter may measured by a second sensor. The first sensor may be different from the second sensor. For example, the first physiological parameter is a heart activity parameter and the first sensor is heart activity sensor; the second physiological parameter is a respiratory parameter and the second sensor is a respiratory sensor. In another embodiment, the first sensor and the second sensor may be the same sensor. For example, the first physiological parameter (e.g., PPI) is measured by a sensor, the second physiological parameter (e.g., heart rate in the form of beats per minute) is derived from the first physiological parameter. For example, each of the first physiological parameter and the second physiological parameter is derived from raw data measured by a sensor.

The psychological stress increases as each of the heart rate variation and the relative heart rate variation (determined based on the reference value of the heart rate variation and may be a difference between the heart rate variation and the reference value of the heart rate variation) decreases. However, even if there is the same heart rate variation or the same relative heart rate variation estimated in two psychological states of the user, there isn't necessarily the same psychological stress sensed by the user in two psychological states.

In the present invention, a second physiological parameter (i.e., another physiological parameter) is taken into account to reduce imprecision of an estimation of the psychological stress determined by one physiological parameter (e.g., heart rate variation). Physiologically, the intensity associated with the internal workload (e.g., heart rate) increases when the psychological stress sensed by the user increases. Therefore, the intensity associated with the internal workload is preferably taken into account to reduce imprecision of an estimation of the psychological stress determined by one physiological parameter (e.g., heart rate variation). The intensity associated with the internal workload may be a heart activity parameter, such as heart rate.

Then, the first physiological parameter and the second physiological parameter (different from the first physiological parameter) may be used together to determine a psychological stress. The first physiological parameter and the second physiological parameter may be simultaneously acquired to used together to determine the psychological stress. The second physiological parameter may temporally correspond to the first physiological parameter.

When the first physiological parameter and the second physiological parameter are used together to determine the psychological stress, the physiological parameter data may have a different reliability in an estimation of the psychological stress. For example, the physiological parameter data having a lower heart rate variation and a higher heart rate (e.g., in the form of beats per minute) is prone to be directed to have a psychological stress, but the physiological parameter data having a higher heart rate variation and a higher heart rate isn't necessarily to be directed to a have a psychological stress. In other words, the reliability in an estimation of the psychological stress of the former physiological parameter data is more than that of the latter physiological parameter data. Therefore, in order to precisely determine the physiological stress based on the physiological parameter data, determining a reliability in an estimation of the psychological stress of the physiological parameter data is needed.

The present invention sets up a plurality of criteria to determine a reliability in an estimation of the psychological stress. Once the criteria are determined, determine a reliability in an estimation of the psychological stress of the physiological parameter data based on a degree of match between the physiological parameter data and a plurality of criteria (step 22). The criteria may be determined based on a first reference value of the first physiological parameter and a second reference value of the second physiological parameter, which can be seen in case (I) and case (II).

Even if the psychological stress is estimated to be the same in an early time and in a later time for a person, the person doesn't necessarily sense the same psychological stress. The person may actually sense the different psychological stress because the reference value of each of at least two physiological parameters used in an estimation of the psychological stress may vary with time. In the present invention, each of the first reference value of the first physiological parameter and the second reference value of the second physiological parameter is determined based on the corresponding history data so as to increase a precision of the estimation of a reliability in an estimation of the psychological stress and further to increase a precision of the estimation of the psychological stress, which will be described hereafter.

In case (I), the reference value of the heart rate variation may be 90 ms and the reference value of the heart rate may be 60 BPM; the criteria may comprise “(A) the heart rate variation is less than 90 ms” and “(B) the heart rate is more than 60 BPM” can be set up to be directed to have a psychological stress; in condition (I-1) of case (I), if the physiological parameter data meets “(A) the heart rate variation is less than 90 ms” and “(B) the heart rate is more than 60 BPM”, the reliability R11 in an estimation of the psychological stress of the physiological parameter data is more; in condition (I-2) of case (I), if the physiological parameter data meets one of “(A) the heart rate variation is less than 90 ms” and “(B) the heart rate variation is more than 60 BPM,” the reliability R12 in an estimation of the psychological stress of the physiological parameter data is less (i.e., the reliability R11 is more than reliability R12). Optionally, if neither of “(A) the heart rate variation is less than 90 ms” and “(B) the heart rate is more than 60 BPM” is met by the physiological parameter data, the physiological parameter data is prone to be directed to have no psychological stress; the psychological stress may be determined by another algorithm or may be not determined/displayed.

In case (II), the reference value of the heart rate variation may be 90 ms, the reference value of the heart rate may be 60 BPM and the reference value of a ratio of the heart rate variation to the heart rate may be 1.5 ms/BPM; the criteria may comprise “(A) the heart rate variation is less than 90 ms,” “(B) the heart rate is more than 60 BPM” and “(C) the ratio of the heart rate variation to the heart rate is less than 1.5 ms/BPM” can be set up to be directed to have a psychological stress. Table 1 is an example listing the reliability in an estimation of the psychological stress based on a degree of match between the physiological parameter data and a plurality of criteria. Optionally, if none of “(A) the heart rate variation is less than 90 ms,” “(B) the heart rate is more than 60 BPM” and “(C) the ratio of the heart rate variation to the heart rate is less than 1.5 ms/BPM is met by the physiological parameter data,” the psychological stress may be determined by another algorithm or may be not determined/displayed.

TABLE 1

| met criteria | Reliability by rank | Reliability by value |
|---|---|---|
| A, B, C | 1 | 90 |
| A, B | 2 | 80 |
| A, C | 3 | 70 |
| B, C | 4 | 60 |
| A | 5 | 50 |
| B | 6 | 40 |
| C | 7 | 30 |

The reliability in an estimation of the psychological stress may be represented in any suitable form. The reliability in an estimation of the psychological stress may be determined based on a degree of match between the physiological parameter data and the criteria. The degree of match between the physiological parameter data and the criteria may depend on a number of a plurality of met criteria, wherein the met criteria are at least one portion of the plurality of criteria which the physiological parameter data meets. Preferably, the number of the met criteria is at least one. For example, the number of the met criteria in condition (I-1) of case (I) is 2 and the number of the met criteria in condition (I-2) of case (I) is 1; therefore, the reliability R11 in an estimation of the psychological stress in condition (I-1) of case (I) is more than the reliability R12 in an estimation of the psychological stress in condition (I-2) of case (I). The reliability in an estimation of the psychological stress may be represented in the form of value or index calculated by any suitable algorithm. For example, the reliability R11 may be 90 in condition (I-1) of case (I) and the reliability R12 may be 60 in condition (I-2) of case (I) if the defined reliability is ranged from 0 to 100.

Once the reliability in the estimation of the psychological stress of the physiological parameter data is determined, determine an indicator of the psychological stress based on the physiological parameter data and the reliability in the estimation of the psychological stress of the physiological parameter data (step 23).

In one embodiment, an algorithm is performed based on the reliability in the estimation of the psychological stress of the physiological parameter data to determine the indicator of the psychological stress for the physiological parameter data.

The following algorithm is a first example of determining the indicator of the psychological stress in case (I); however, the present invention is not limited to this case.

S(t)=f1(X(t),Y(t),R(t))

S(t)=f1(X(t),Y(t),R(0)|_{R(t)=R11}, if R(t)=R11 at time point t

S(t)=f1(X(t),Y(t),R(0)|_{R(t)=R12}, if R(t)=R12 at time point t

S(t) is the psychological stress at the time point t (or at the current time), X(t) is the first physiological parameter of the physiological parameter data at the time point t, Y(t) is the second physiological parameter of the physiological parameter data at the time point t, R(t) is the reliability in the estimation of the psychological stress of the physiological parameter data at the time point t, function f1 is chosen/adjusted according to the observation of the physiological phenomenon.

The following algorithm is a second example of determining the indicator of the psychological stress in case (I); however, the present invention is not limited to this case.

S(t)=f2(X(t),Y(t)), if R(t)=R11 at time point t

S(t)=f3(X(t),Y(t)), if R(t)=R12 at time point t

S(t) is the psychological stress at the time point t (or at the current time), X(t) is the first physiological parameter of the physiological parameter data at the time point t, Y(t) is the second physiological parameter of the physiological parameter data at the time point t, R(t) is the reliability in the estimation of the psychological stress of the physiological parameter data at the time point t, each of function f2 and function f3 is chosen/adjusted according to the observation of the physiological phenomenon.

In the present invention, an algorithm is performed based on the reliability in an estimation of the psychological stress of the physiological parameter data to determine the psychological stress for the physiological parameter data such that the estimation difference between the psychological stress determined in the higher reliability in an estimation of the psychological stress and the psychological stress determined in the lower reliability in an estimation of the psychological stress can meet the actual difference of the psychological stress more. Therefore, a modified algorithm determined based on the reliability in an estimation of the psychological stress can increase a precision of the estimation of the psychological stress.

In a further embodiment, determining the indicator of the psychological stress comprises: modifying at least one of a first relative physiological parameter and a second relative physiological parameter based on the reliability in the estimation of the psychological stress of the physiological parameter data to determine at least one modified physiological parameter and determining the indicator of the psychological stress based on the at least one modified physiological parameter. The first relative physiological parameter may be a first difference between the first physiological parameter and the first reference value of the first physiological parameter, and the second relative physiological parameter may be a second difference between the second physiological parameter and the second reference value of the second physiological parameter; however, the present invention is not limited to this case. One (or each) of the at least one modified physiological parameter may be determined based on a combination of a weighting factor and corresponding one of the first relative physiological parameter and the second relative physiological parameter; the weighting factor is adjusted based on the reliability in the estimation of the psychological stress of the physiological parameter data. One (or each) of the at least one modified physiological parameter may a product of a weighting factor and corresponding one of the first relative physiological parameter and the second relative physiological parameter.

The following algorithm is a third example of determining the indicator of the psychological stress in case (I); however, the present invention is not limited to this case.

$$\begin{matrix}S ( t ) = f 4 ( X ( t ) , Y ( t ) ) , if R ( t ) = R 11 at time point t \\ = f {4}^{′} ( X ( t ) - A , Y ( t ) - B ) \\ = c 1 * [ ( X ( t ) - A ) * F 1 ] + c 2 * [ ( Y ( t ) - B ) * F 2 ) ] + c 3\end{matrix} \\ \begin{matrix}S ( t ) = f 4 ( X ( t ) , Y ( t ) ) , if R ( t ) = R 12 at time point t \\ = f {4}^{′} ( X ( t ) - A , Y ( t ) - B ) \\ = c 1 * [ ( X ( t ) - A ) * F 1 ] + c 2 * [ ( Y ( t ) - B ) * F 2 ) ] + c 3\end{matrix} \\ or \\ \begin{matrix}S ( t ) = f 5 ( X ( t ) , Y ( t ) ) , if R ( t ) = R 12 at time point t \\ = f {5}^{′} ( X ( t ) - A , Y ( t ) - B ) \\ = c 4 * [ ( X ( t ) - A ) * F 1 ] + c 5 * [ ( Y ( t ) - B ) * F 2 ) ] + c 6\end{matrix}$$

S(t) is the psychological stress at the time point t (or at the current time) and may be ranged from 0 to 100, X(t) is the first physiological parameter of the physiological parameter data at the time point t, A is the first reference value of the first physiological parameter, (X(t)—A) is the first relative physiological parameter, Y(t) is the second physiological parameter of the physiological parameter data at the time point t, B is the second reference value of the second physiological parameter, (Y(t)—B) is the second relative physiological parameter, R(t) is the reliability in the estimation of the psychological stress of the physiological parameter data at the time point t, each of function f4, function f4′, function f5 and function f5′ is chosen/adjusted according to the observation of the physiological phenomenon (e.g., the psychological stress increases as the heart rate variation decreases and the psychological stress increases as the heart rate increases in function f4), each of c1 to c6 is a coefficient adjusted according to the observation of the physiological phenomenon, each of F1 and F2 is a weighting factor adjusted based on the reliability in the estimation of the psychological stress of the physiological parameter data.

In condition (I-2) of case (I), if the physiological parameter data doesn't meet “(A) the heart rate variation is less than 90 ms,” the weighting factor F1 is adjusted (i.e., the weighting factor F1 in R(t)=R11 is different from the weighting factor F1 in R(t)=R12). At this time, the reliability in the estimation of the psychological stress of the physiological parameter data depends on (e.g., a positive or a negative of) the relative heart rate variation (i.e., first relative physiological parameter) and thus compensating the reliability in the estimation of the psychological stress of the physiological parameter by modifying the relative heart rate variation, which contributes to reduce an imprecision of the estimation of the psychological stress; therefore, in a further embodiment, the present invention modifies the relative heart rate variation by adjusting the weighting factor F1 corresponding to the heart rate variation to increase an precision of the estimation of the psychological stress.

In condition (I-2) of case (I), if the physiological parameter data doesn't meet “(B) the heart rate is more than 60 BPM,” the weighting factor F2 is adjusted (i.e., the weighting factor F2 in R(t)=R11 is different from the weighting factor F2 in R(t)=R12). At this time, the reliability in the estimation of the psychological stress of the physiological parameter data depends on (e.g., a positive or a negative of) the relative heart rate (i.e., second relative physiological parameter) and thus compensating the reliability in the estimation of the psychological stress of the physiological parameter by modifying the relative heart rate, which contributes to reduce an imprecision of the estimation of the psychological stress; therefore, in a further embodiment, the present invention modifies the relative heart rate by adjusting the weighting factor F2 corresponding to the heart rate to increase an precision of the estimation of the psychological stress.

Even if the psychological stress is estimated to be the same in an early time and in a later time for a person, the person doesn't necessarily sense the same psychological stress. The person may actually sense the different psychological stress because the reference value of each of at least two physiological parameters used in an estimation of the psychological stress may vary with time. In the present invention, the reference value of each of at least two physiological parameters is determined based on the corresponding history data to increase a precision of the estimation of the psychological stress. The first reference value of the first physiological parameter may be determined based on the first history data associated with the first physiological parameter and the second reference value of the second physiological parameter may be determined based on the second history data associated with the second physiological parameter.

The first reference value of the first physiological parameter may be an average of the values of the first physiological parameter in the first history data associated with the first physiological parameter. The second reference value of the second physiological parameter may be an average of the values of the second physiological parameter in the second history data associated with the second physiological parameter. The first reference value of the first physiological parameter may be a median of the values of the first physiological parameter in the first history data associated with the first physiological parameter. The second reference value of the second physiological parameter may be a median of the values of the second physiological parameter in the second history data associated with the second physiological parameter. However, the present invention is not limited to above cases.

The physiological parameter data may exclude a portion of initial physiological parameter data associated with exercise and a recovery from the exercise. The physiological parameter data may exclude a portion of initial physiological parameter data associated with exercise. The data associated with exercise may be data with an activity intensity more than a threshold.

The above disclosure is related to the detailed technical contents and inventive features thereof. People skilled in the art may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A method for determining psychological stress, the method comprising:
acquiring physiological parameter data measured by at least one sensor, wherein the physiological parameter data comprises a first physiological parameter and a second physiological parameter temporally corresponding to the first physiological parameter;
determining, by a processing unit, a reliability in an estimation of the psychological stress of the physiological parameter data based on a degree of match between the physiological parameter data and a plurality of criteria, wherein the plurality of criteria are determined based on a first reference value of the first physiological parameter and a second reference value of the second physiological parameter; and
determining, by the processing unit, an indicator of the psychological stress based on the physiological parameter data and the reliability in the estimation of the psychological stress of the physiological parameter data.

2. The method according to claim 1, wherein the degree of match between the physiological parameter data and the plurality of criteria depends on a number of a plurality of met criteria, wherein the plurality of met criteria are at least one portion of the plurality of criteria which the physiological parameter data meets.

3. The method according to claim 1, wherein determining the indicator of the psychological stress comprises that an algorithm is performed based on the reliability in the estimation of the psychological stress of the physiological parameter data to determine the indicator of the psychological stress for the physiological parameter data.

4. The method according to claim 1, wherein determining the indicator of the psychological stress comprises:
modifying at least one of a first relative physiological parameter and a second relative physiological parameter based on the reliability in the estimation of the psychological stress of the physiological parameter data to determine at least one modified physiological parameter, wherein the first relative physiological parameter is determined based on the first reference value of the first physiological parameter and the second relative physiological parameter is determined based on the second reference value of the second physiological parameter; and
determining the indicator of the psychological stress based on the at least one modified physiological parameter.

5. The method according to claim 4, wherein the first relative physiological parameter is a first difference between the first physiological parameter and the first reference value of the first physiological parameter, and the second relative physiological parameter is a second difference between the second physiological parameter and the second reference value of the second physiological parameter.

6. The method according to claim 5, wherein one of the at least one modified physiological parameter is determined based on a combination of a weight factor and corresponding one of the first relative physiological parameter and the second relative physiological parameter, wherein the weighting factor is adjusted based on the reliability in the estimation of the psychological stress of the physiological parameter data.

7. The method according to claim 1, further comprising displaying, by a displaying unit, the indicator of the psychological stress.

8. The method according to claim 4, further comprising displaying, by a displaying unit, the first reference value of the first physiological parameter, the second reference value of the second physiological parameter and the indicator of the psychological stress.

9. The method according to claim 1, wherein the first reference value of the first physiological parameter is determined based on first history data associated with the first physiological parameter and the second reference value of the second physiological parameter is determined based on second history data associated with the second physiological parameter.

10. The method according to claim 1, wherein the physiological parameter data excludes a portion of initial physiological parameter data associated with exercise and a recovery from the exercise.

11. The method according to claim 1, wherein the first physiological parameter is associated with a variation of a heart activity parameter.

12. The method according to claim 1, wherein the second physiological parameter is an intensity associated with an internal workload.

13. The method according to claim 12, wherein the second physiological parameter is a heart activity parameter.

14. The method according to claim 13, wherein the second physiological parameter is a heart rate.

15. The method according to claim 1, wherein the at least one sensor comprises a first sensor and a second sensor different from the first sensor, wherein the first physiological parameter is measured by a first sensor and the second physiological parameter is measured by a second sensor.

16. The method according to claim 2, wherein the number of the plurality of met criteria is at least one.

17. A method for determining psychological stress, the method comprising:
acquiring physiological parameter data measured by at least one sensor, wherein the physiological parameter data comprises a first physiological parameter and a second physiological parameter temporally corresponding to the first physiological parameter, wherein the first physiological parameter is a heart rate variation and the second physiological parameter is a heart rate;
determining, by a processing unit, a reliability in an estimation of the psychological stress of the physiological parameter data based on a degree of match between the physiological parameter data and a plurality of criteria, wherein the plurality of criteria are determined based on a first reference value of the first physiological parameter and a second reference value of the second physiological parameter; and
determining, by the processing unit, an indicator of the psychological stress based on the physiological parameter data and the reliability in the estimation of the psychological stress of the physiological parameter data.

18. The method according to claim 17, wherein the first reference value of the first physiological parameter is determined based on first history data associated with the first physiological parameter and the second reference value of the second physiological parameter is determined based on second history data associated with the second physiological parameter.

19. A method for determining psychological stress, the method comprising:
acquiring physiological parameter data measured by at least one sensor, wherein the physiological parameter data comprises a first physiological parameter and a second physiological parameter temporally corresponding to the first physiological parameter, wherein the first physiological parameter is a heart rate variation and the second physiological parameter is a heart rate;
determining, by a processing unit, a reliability in an estimation of the psychological stress of the physiological parameter data based on a degree of match between the physiological parameter data and a plurality of criteria, wherein the plurality of criteria are determined based on a first reference value of the first physiological parameter and a second reference value of the second physiological parameter;
modifying at least one of a first relative physiological parameter and a second relative physiological parameter based on the reliability in the estimation of the psychological stress of the physiological parameter data to determine at least one modified physiological parameter, wherein the first relative physiological parameter is a first difference between the first physiological parameter and the first reference value of the first physiological parameter, and the second relative physiological parameter is a second difference between the second physiological parameter and the second reference value of the second physiological parameter, wherein one of the at least one modified physiological parameter is determined based on a combination of a weighting factor and corresponding one of the first relative physiological parameter and the second relative physiological parameter, wherein the weighting factor is adjusted based on the reliability in the estimation of the psychological stress of the physiological parameter data; and
determining the indicator of the psychological stress based on the at least one modified physiological parameter.

20. The method according to claim 19, wherein the first reference value of the first physiological parameter is determined based on first history data associated with the first physiological parameter and the second reference value of the second physiological parameter is determined based on second history data associated with the second physiological parameter.
